(19) United States
(12) Patent Application Publication
(10) Pub. No.: US 2003/0073984 A1
(43) Pub. Date: Apr. 17, 2003

(54) Corneal-ablation-data determining apparatus and a corneal surgery apparatus

(75) Inventor: Naoyuki Maeda, Minoo-shi (JP)

Correspondence Address: OLIFF & BERRIDGE, PLC, P.O. BOX 19928, ALEXANDRIA, VA, 22320, US

(73) Assignee: NIDEK CO., LTD., Gamagori-shi (JP)

(21) Appl. No.: 10/268,992

(22) Filed: Oct. 11, 2002

(30) Foreign Application Priority Data
Oct. 12, 2001 (JP) 2001-315441

(51) Int. Cl. A61F 9/007

(52) U.S. Cl. 606/5; 606/10; 606/13

(57) ABSTRACT

A corneal-ablation-data determining apparatus and a corneal surgery apparatus capable of performing corneal ablation surgery efficiently while improving an aberration of a patient's eye. The corneal-ablation-data determining apparatus for obtaining ablation data for corneal ablation surgery to correct a refractive error by ablating a cornea, the apparatus comprising an input device for inputting measurement data of a patient's eye and size data of an ablation zone and a calculation device for calculating ablation data including aberration improvement data for the patient's eye based on the inputted measurement data and size data, wherein the calculation device divides the ablation zone into a central zone and a peripheral zone outside the central zone, then calculates ablation data for a component not being a non-spherical component in the central zone, and calculates ablation data for a non-spherical component in at least part of the peripheral zone, including the aberration improvement data.

### BACKGROUND OF THE INVENTION

1. Field of the Invention

The present invention relates to a corneal-ablation-data determining apparatus for calculating corneal ablation data for corneal ablation surgery in order to correct a refractive error by ablating a cornea and a corneal surgery apparatus for performing the corneal ablation surgery based on the corneal ablation data.

2. Description of Related Art

Conventionally, there is known a corneal surgery apparatus which ablates a cornea with a laser beam and changes the shape of the cornea in order to correct a refractive error of a patient's eye. Methods for ablating a cornea with this kind of the apparatus include a method (a one-shot method) for performing one-shot irradiation of a laser beam for ablation, wherein a beam cross section is vertical to a laser irradiation optical axis and has a large circular shape (a large spot), a method (a slit scan method) for irradiating and scanning a laser beam in at least one direction for ablation, wherein a beam cross section is rectangular, a method (a spot scan method) for two-dimensionally irradiating and scanning a laser beam for ablation, wherein a beam cross section has a small circular shape (a small spot), and other methods.

In addition, as for the one-shot method and the slit scan method, Japanese Patent Application Unexamined Publication No. Hei 09-266925 corresponding to U.S. Pat. No. 5,906,608 suggests a method for ablation by irradiating a laser beam of which cross section is limited to a circular or rectangular small zone by a small aperture which is circular, rectangular or the like. By these methods, a non-spherical component (mentioned as a rotationally symmetrical component and a linearly symmetrical component which are not spherical nor toric, and an asymmetric component in the present specification) may be ablated to improve (correct) an aberration of an eye.

However, in the case of the ablation of the non-spherical component, the method of irradiating the laser beam limited to the small zone for ablation has a disadvantage of the irradiation time becoming longer. As the irradiation time becomes longer, the laser irradiation optical axis aligned with the patient's eye becomes easy to decenter, and errors in the aberration improvement are generated frequently. Particularly, the center part of the cornea is influenced largely.

### SUMMARY OF THE INVENTION

An object of the invention is to overcome the problems described above and to provide a corneal-ablation-data determining apparatus and a corneal surgery apparatus for performing corneal ablation surgery efficiently while improving an aberration of a patient's eye.

To achieve the objects and in accordance with the purpose of the present invention, a corneal-ablation-data determining apparatus comprises input means for inputting measurement data of a patient's eye and size data of an ablation zone and calculation means for calculating ablation data including aberration improvement data for the patient's eye based on the inputted measurement data and size data, wherein the calculation means divides the ablation zone into a central zone and a peripheral zone outside the central zone, then calculates ablation data for a component not being a non-spherical component in the central zone, and calculates ablation data for a non-spherical component in at least part of the peripheral zone, including the aberration improvement data.

In another aspect of the present invention, a corneal surgery apparatus comprises laser irradiation means for irradiating a laser beam to a cornea, input means for inputting measurement data of a patient's eye and size data of an ablation zone, calculating means for calculating ablation data including aberration improvement data for the patient's eye based on the inputted measurement data and size data, and control means for controlling the laser irradiation means based on the obtained ablation data, wherein the calculating means divides the ablation zone into a central zone and a peripheral zone outside the central zone, then calculates ablation data for a component not being a non-spherical component in the central zone, and calculates ablation data for a non-spherical component in at least part of the peripheral zone, including the aberration improvement data.

Yet, in another aspect of the present invention, a corneal surgery apparatus comprises laser irradiation means for irradiating a laser beam to the cornea, input means for inputting ablation data including aberration improvement data for a patient's eye, the ablation data being obtained based on measurement data of the patient's eye, calculating means for correcting the inputted ablation data and control means for controlling the laser irradiation means based on the corrected ablation data, wherein the calculating means divides ablation zone into a central zone and a peripheral zone outside the central zone, then calculates ablation data for a component not being a non-spherical component in the central zone, and calculates ablation data for a non-spherical component in at least part of the peripheral zone, including the aberration improvement data.

Additional objects and advantages of the invention are set forth in the description which follows, are obvious from the description, or may be learned by practicing the invention. The objects and advantages of the invention may be realized and attained by the apparatus for determining corneal ablation data and the corneal surgery apparatus in the claims,

### BRIEF DESCRIPTION OF THE DRAWINGS

The accompanying drawings, which are incorporated in and constitute a part of this specification, illustrate embodiments of the present invention and, together with the description, serve to explain the objects, advantages and principles of the invention. In the drawings,

FIG. 1 is a view showing a schematic configuration block diagram of a corneal surgery apparatus system consistent with the present invention;

FIG. 2 is a view showing a schematic configuration of an optical system and a control system of the corneal surgery apparatus;

FIG. 3 is a view showing a schematic configuration of a dividing aperture plate and a dividing shutter;

FIG. 4 is a view showing an example of a dividing pattern of an ablation zone;

FIG. 5 is a view showing an example of a display of ablation data; and

FIG. 6 is a view showing an example of a dividing pattern of an ablation zone.

### DETAILED DESCRIPTION OF THE PREFERRED EMBODIMENTS

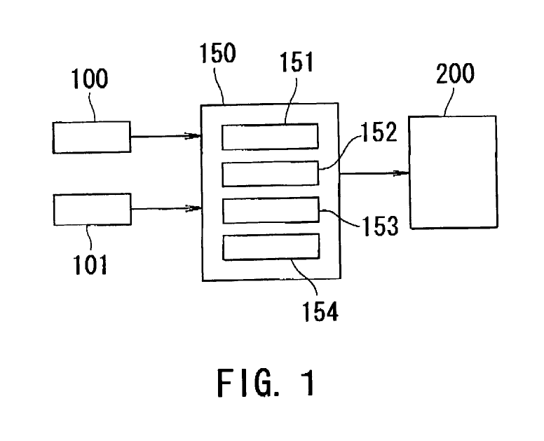

A detailed description of one preferred embodiment of a corneal-ablation-data determining apparatus and a corneal surgery apparatus embodied by the present invention are provided below with reference to the accompanying drawings. FIG. 1 is a view showing a schematic configuration block diagram of a corneal surgery apparatus system consistent with the present invention.

An apparatus 100 for measuring a distribution of eye refractive power of a patient's eye and an apparatus 101 for measuring a corneal shape of the patient's eye obtain measurement data of the patient's eye in order to determine corneal ablation data. The measurement apparatus 100 may be the following one (see Japanese Patent Application Unexamined Publication No. Hei 10-108837 corresponding to U.S. Pat. No. 5,907,388). That one is provided with an optical system for projecting and scanning a slit light bundle onto a fundus of the patient's eye, and a detecting optical system having a plurality of pairs of photodetectors being disposed diametrically with respect to an optical axis at a position generally conjugate with the cornea in a meridian direction corresponding to a slit direction of the slit light bundle. In addition, the apparatus also obtains a wide range of a distribution of eye refractive power varying in the meridian direction based on a phase difference signal outputted from each photodetector by rotating the slit light bundle and the photodetectors on the optical axis in synchronization with each other. Further, the measurement apparatus 101 may be the one which projects a plurality of annular placido rings to the cornea of the patient's eye and detects images of the rings in order to obtain a wide range of a distribution of corneal curvature.

A calculating device 150 for calculating the corneal ablation data is provided with a calculation unit 151, an input unit 152, a display unit 153, an output unit 154 and the like. A commercial personal computer may be used instead of this device. The measurement data obtained by the measurement apparatuses 100 and 101 are inputted to the calculation unit 151 using the input unit 152 via cable communication or storage media like a floppy disc. In addition, size data of the ablation zone and the like are also inputted to the calculation unit 151 by the input unit 152. The display unit 153 displays determined ablation data graphically.

A corneal surgery apparatus 200 ablates the cornea with a laser beam. The ablation data obtained by the calculating device 150 is outputted from the output unit 154 and inputted to the surgery apparatus 200 via cable communication or storage media like a floppy disc.

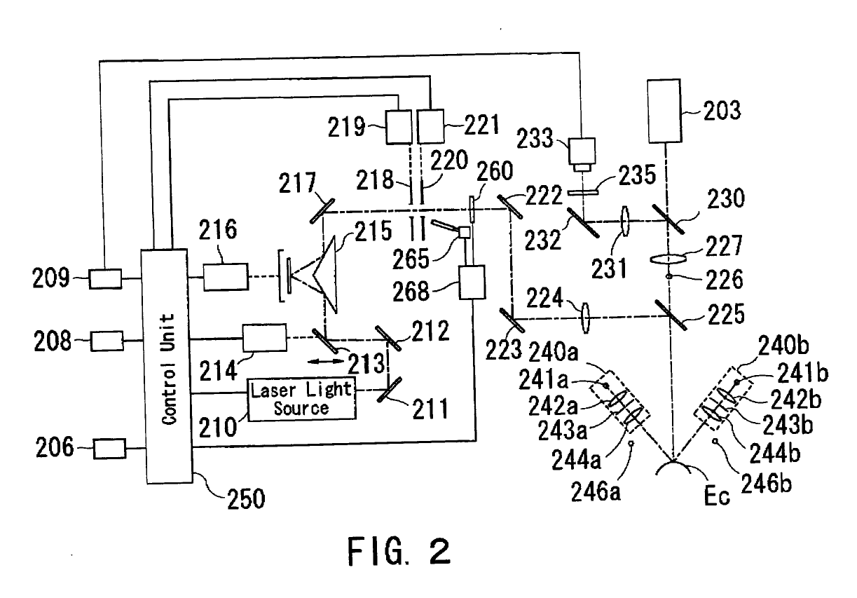

FIG. 2 is a view showing a schematic configuration of an optical system and a control system of the corneal surgery apparatus 200. A laser light source 210 emits an excimer laser beam having a wavelength of 193 nm. The laser beam emitted from the laser light source 210 is reflected by mirrors 211 and 212, and is further reflected by a plane mirror 213. A mirror driving unit 214 moves (translates) the mirror 213 in the direction of the arrow shown in FIG. 2, and the mirror 213 moves (scans) the laser beam in the direction of the Gaussian distribution (see Japanese Patent Application Unexamined Publication No. Hei 04-242644 corresponding to U.S. Pat. No. 5,507,799)

An image rotator 215 is rotatably driven on a central optical axis (a laser irradiation optical axis) by an image rotator driving unit 216, and rotates the laser beam on the central optical axis (see Japanese Patent Application Unexamined Publication No. Hei 06-114083 corresponding to U.S. Pat. No. 5,637,109). A mirror 217 changes the direction of the laser beam.

A variable circular aperture 218 limits an ablation zone to a circular shape, and its opening diameter is changed by an aperture driving unit 219. A variable slit aperture 220 limits the ablation zone to a slit shape, and its opening width and the direction of its slit aperture are changed by an aperture driving unit 221. Mirrors 222 and 223 change the direction of the laser beam. A projecting lens 224 projects the circular aperture 218 and the slit aperture 220 to a cornea Ec of the patient's eye.

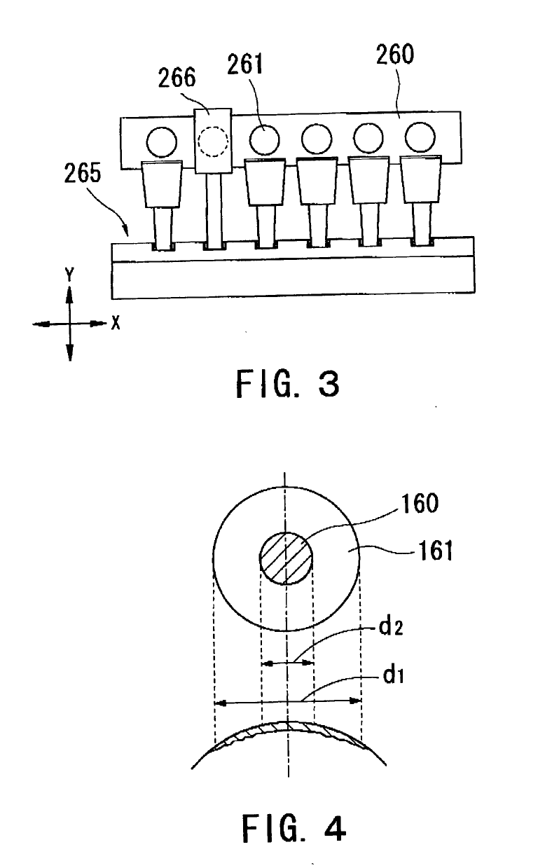

In addition, a dividing aperture plate 260 is disposed insertably and removably on an optical path between the slit aperture 220 and the mirror 222. The dividing aperture plate 260 further limits the ablation zone in combination with a dividing shutter 265. The dividing aperture plate 260 and the dividing shutter 265 are used when ablating an asymmetric component of the cornea Ec. When the dividing aperture 260 is viewed from the light source 210 side, six circular small apertures 261 of the same size are aligned as shown in FIG. 3, One or more shutter plates 266 of the dividing shutter 265 cover and uncover the small apertures 261 selectively, thereby enabling the ablation zone to be further limited to a smaller zone for laser irradiation. Incidentally, each small aperture 261 is provided with a correcting optical system for correcting an intensity distribution of the laser beam which has been transmitted through its opening. According to the correcting optical system, an energy distribution of the laser beam of a circular spot irradiating the cornea Ec is corrected to be high at the center and low at the periphery. The energy distribution is preferably the Gaussian distribution. Further, the dividing aperture plate 260 and the dividing shutter 265 can be moved by a driving unit 268 within a plane vertical to the central optical axis.

A dichroic mirror 225 has a property of reflecting the excimer laser beam of 193 nm and transmitting visible light and infrared light, and the laser beam which has been transmitted through the projecting lens 224 is reflected by the dichroic mirror 225 to be directed to the cornea Ec.

Placed above the dichroic mirror 225 are a visible fixation light 226, an objective lens 227 and a microscope unit 203. A mirror 230 is disposed between binocular optical paths of the microscope unit 203 (on an optical axis of the objective lens 227). An image forming lens 231, a mirror 232, an infrared transmission filter 235 and a CCD camera 233 which is sensitive to the infrared region are disposed on an optical path at a reflecting side of the mirror 230. The lens 227, the mirror 230, the mirror 232, the filter 235 and the camera 233 constitute an optical system for picking up an image of an anterior-segment of the patient's eye and detecting a position of an eyeball. The camera 233 connects to a computer 209. Infrared light sources 246a and 246b are for illumination of an anterior-segment of an eye.

Placed below the dichroic mirror 225 symmetrically across the optical axis of the lens 227 are slit projection optical systems 240a and 240b. The slit projection optical systems 240a and 240b are composed of illumination lamps 241a and 241b which emit visible light, condenser lenses 242a and 242b, slit plates 243a and 243b each having a cross-shaped slit, and projecting lenses 244a and 244b, respectively. The slit plates 243a and 243b are in a conjugate positional relation to the cornea Ec relative to the lenses 244a and 244b, and an image of the cross-shaped slit is formed at all times at a focal point on the optical axis of the lens 227 (see Japanese Patent Application Unexamined Publication No. Hei 06-047001 corresponding to U.S. Pat. No. 5,562,656).

A control unit 250 controls the laser light source 210, each of the driving units 214, 216, 219, 221 and 268, and the like. The control unit 250 connects to a toot switch 208, a controller 206 where a variety of operation switches are disposed, and the computer 209. The computer 209 is provided with an input unit for inputting a surgical condition and the like and a display unit, and performs calculation, display, storage and the like of control data.

Incidentally, it is preferable that the surgery apparatus 200 has an eye tracking function (a function for tracking the patient's eye in order to adjust a laser irradiation position in case that the patient's eye moves during alignment or laser irradiation), and the one described in Japanese Patent Application Unexamined Publication No. Hei 09-149914 corresponding to U.S. Pat. No. 6,159,202, or the like may be used. An output of the camera 233 is utilized for detecting an eyeball position for the eye tracking.

Next, an operation of the corneal surgery apparatus system as above will be described. The measurement data obtained in the measuring apparatuses 100 and 101 are inputted to the calculating unit 151 using the input unit 152. Further, the size data of the ablation zone and size data for dividing the ablation zone are inputted to the calculation unit 151 using the input unit 152, wherein the ablation zone is divided into a zone where a component not being a non-spherical component is ablated and a zone where a non-spherical component is ablated

FIG. 4 is a view showing an example of a dividing pattern of the ablation zone. Firstly, a size d1 of an optical zone is inputted as the size of the ablation zone. Since the size d1 of φ7 mm is generally used, that size may be set previously, however, it is preferable to set a size larger than the pupil size of the patient's eye in scotopic vision. The size may also be obtained by picking up the image of the anterior-segment of the eye at the measurement of an eye refractive power in scotopic vision, or before or after the measurement, and measuring the pupil size from the image.

Secondly, a sized d2 of a central zone 160 is inputted. The zone 160 is a zone where the component not being a non-spherical component is ablated. The size may he set previously at φ2 to 3 mm, but preferably the pupil size of the patient's eye in photopic vision. That size may also be obtained by picking up the image of the anterior-segment of the eye at the measurement of the corneal shape in photopic vision, or before or after the measurement, and measuring the pupil size from the image. The zone 160 is a zone for ensuring day-vision. The size d2 of the zone 160 is inputted to separate a peripheral zone 161 as a zone where the non-spherical component is ablated to improve an aberration of the eye. The peripheral zone 161 is a zone for ensuring night-vision. Incidentally, a transition zone connecting smoothly the ablation zone and the non-ablation zone is placed outside the ablation zone (outside the zone 161), though the transition zone is not illustrated in FIG. 4.

The calculation unit 151 calculates the corneal ablation data based on the inputted data. Firstly, three-dimensional data on a corneal shape is obtained from distribution data on corneal curvature obtained by the measurement, and converted into distribution data on corneal refractive power according to the Snell's law. Secondly, distribution data on eye refractive power obtained by the measurement is converted into those at the corneal position. These data are used to express a refractive power which is necessary to improve the aberration and to achieve emmetropia (or a non-aberration eye having a specific refractive power) in a form of a corneal refractive power, thereby obtaining distribution data on equivalent emmetropic corneal refractive power. The obtained data is converted into distribution data on corneal curvature, i.e. to three-dimensional data on a corneal shape, according to the Snell's law. Then, the size data of the ablation zone (the optical zone) is provided, and the obtained three-dimensional data on a corneal shape is subtracted from the three-dimensional data on a corneal shape obtained at the corneal shape measurement, thereby obtaining ablation amount distribution data for making the whole ablation zone emmetropic.

As for the zone 160, the ablation amount distribution data are corrected to an averaged value for ablating the component not being a non-spherical component (a spherical component or a toric component). Besides, each averaged value of the corneal curvature distribution data and the eye refractive power distribution data which are obtained by the measurement may be used to calculate the ablation amount distribution data according to the above-mentioned calculation. On the other hand, as for the zone 161, the ablation amount distribution data for ablating the non-spherical component are calculated according to the above-mentioned calculation. Incidentally, when a connecting part of the zone 160 and the zone 161 becomes discontiguous, it is preferable to use ablation data which connect both zones smoothly.

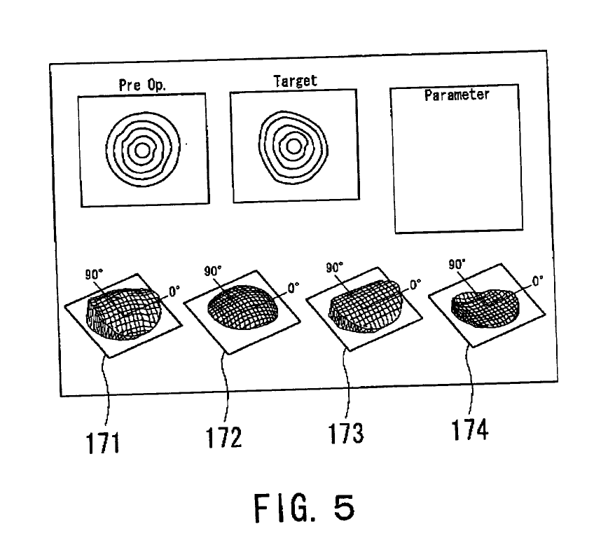

Besides, the ablation amount distribution data are calculated as an entire ablation amount distribution data. In addition, in order to enable efficient corneal surgery, the ablation amount distribution data are calculated while being divided into those for a rotationally symmetrical component, a linearly symmetrical component and an asymmetric component. Each of the ablation amount distribution data is displayed graphically on the display unit 153 in three-dimensional shape such as a bird's eye view or the like, and FIG. 5 is an example thereof. A graphic display 171 shows an entire ablation amount distribution map. A graphic display 172 shows the ablation amount distribution map of only the rotationally symmetrical component, a graphic display 173 shows that of only the linearly symmetrical component, and a graphic display 174 shows that of only the asymmetric component.

Incidentally, in the above-mentioned description, the apparatus 100 obtains the eye refractive power distribution as the measurement data of the patient's eye for obtaining the ablation data. However, an apparatus for obtaining a wave aberration distribution may also be used (see U.S. Pat. No. 6,086,204). Since the refractive power distribution may be replaced with a form of the wave aberration distribution, it can be said that both of them are equivalent. The ablation amount distribution data may simply be calculated from the wave aberration distribution, however, more accuracy is ensured by calculating in relation to the three-dimensional data on a corneal shape.

Next, the operation of the surgery apparatus 200 will be described. The ablation data obtained by the calculating device 150 are outputted from the output unit 154 and inputted to the computer 209. The computer 209 calculates control data for controlling each driving unit of the optical system of the surgery apparatus 200 based on the inputted ablation data, and outputs the control data to the control unit 250.

A keratorefractive surgery using the surgery apparatus 200 will be described hereinafter; in particular, myopic correction is taken as an example. A surgeon aligns the pupillar center with the central optical axis by way of the microscope unit 203 so that an unillustrated reticle and the pupillar center of the patient's eye have a predetermined relation with each other. In addition, for adjusting a working distance, slit images projected from the slit projection optical systems 240a and 240b are observed, and the slit images of both systems are made to coincide with each other at the center. Once the foot switch 208 has been pushed after the completion of the alignment, the control unit 250 controls each driving unit to ablate the cornea Ec, as described below, based on the control data according to each ablation data for the rotationally symmetrical component, the linearly symmetrical component and the asymmetric component.

In the myopic correction, and in the case of ablating the rotationally symmetrical component, the control unit 250 limits the ablation zone with the circular aperture 218, and moves (scans) the laser beam in the direction of the Gaussian distribution while moving the mirror 213 sequentially. Then, every time the laser beam provides one scan, the movement (scanning) direction of the laser beam is changed by the rotation of the image rotator 215 (e.g. three directions having a spacing of 120 degrees) to perform approximately uniform ablation of the zone limited by the circular aperture 218. This operation is performed every time the opening diameter of the circular aperture 218 is changed sequentially. And then, in the range of the zone 160, the opening diameter of the circular aperture 218 is changed to ablate the spherical component, and in the range of the zone 161, the opening diameter of the circular aperture 218 is changed to ablate the non-spherical component.

In the case of ablating the linearly symmetrical component, the control unit 250 fixes the opening diameter of the circular aperture 218 while aligning it with the optical zone, and changes the opening width of the slit aperture 220. In addition, the direction of the slit opening of the slit aperture 220 is adjusted by the driving unit 221 in order that the slit opening width changes in the steepest meridian direction. As is the case with the aforementioned ablation of the rotationally symmetrical component, during laser irradiation, the mirror 213 is moved sequentially to move (scan) the laser beam in the direction of the Gaussian distribution. Every time one scan is provided with the laser beam, the movement (scanning) direction of the laser beam is changed by the rotation of the image rotator 215, and the zone limited by the slit aperture 220 is ablated approximately uniformly. And then, this operation is repeated while changing the opening width of the slit aperture 220 in order.

In the case of ablating the partially asymmetric component, the control unit 250 disposes the dividing aperture plate 260 on the optical path, and adjusts the position of the small apertures 261 of the dividing aperture plate 260, while releasing and shielding the small apertures 261 selectively by driving the dividing shutter 265. When the laser beam is moved (scanned) by the movement of the mirror 213, the laser beam is transmitted through the released small apertures 261, whereby the cornea Ec is irradiated only with the laser beam limited to a small zone. The ablation at each position is conducted by controlling irradiation time or the number of scanning. This ablation is performed only in the zone 161.

According to the laser irradiation control as above, the spherical component or the toric component are ablated in the zone 160 shown in FIG. 4, and the non-spherical component is ablated in order to improve the aberration only in the zone 161. Since the non-spherical component is not ablated in the zone 160, an irradiation deviation has a reduced influence on the zone 160. Further, since the improvement of the aberration of the eye mainly depends on the periphery, even if the ablation of the non-spherical component for improving the aberration is not performed in the zone 160, the refractive correction may be performed with approximate accuracy. Furthermore, since the partial ablation with the laser beam of the small zone is not permitted to be performed in the zone 160, the surgery may be performed efficiently while the total surgery time is shortened.

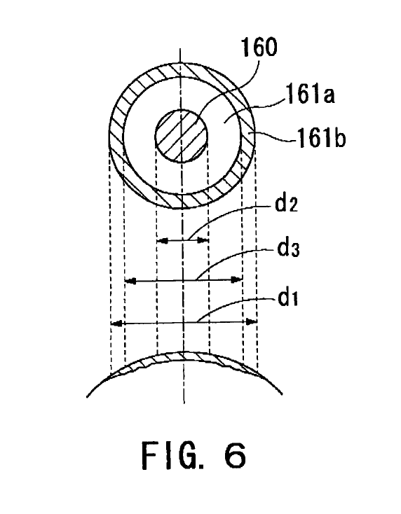

Next, a modified embodiment will be described along with FIG. 6, where a zone in which the non-spherical component is ablated is changed. In this embodiment, the zone 161 shown before in FIG. 4 is further divided into a first zone 161a and a second zone 161b outside the first zone 161a, and the non-spherical component is ablated in the first zone 161a.

In FIG. 6, the ablation zone and the zone 160 are the same as those of the previous embodiment (FIG. 4). In addition, the component not being a non-spherical component is ablated in the second zone 161b of a size d1-d3. The zone 161b is mainly used at nighttime when the pupil expands.

On the other hand, the first zone 161a of a size d3-d2 is used in a little dark place. An inner size d2 of the first zone 161a (i.e. the size d2 of the zone 160) is preferably a pupil size of the patient's eye in photopic vision. An outer size d3 is preferably smaller than the pupil size of the patient's eye in scotopic vision. Incidentally, the size d3 may generally be within a range of φ3 to 6 mm.

The measurement data obtained at the measurement apparatuses 100 and 101 are inputted to the calculation unit 151 using the input unit 152. In addition, the sizes d1, d2 and d3 mentioned above are inputted (or predetermined values are called up from a memory to be inputted automatically). With the same calculation as that described above, the calculating unit 151 calculates the ablation amount distribution data to ablate the component not being a non-spherical component in the zone 160 and the second zone 161b, and calculates the ablation amount distribution data to ablate the non-spherical component for the first zone 161a. Further, the obtained ablation data are inputted to the computer 209 so that the non-spherical component is ablated in the first zone 161a, and that the component not being a non-spherical component is ablated in the zone 160 and the second zone 161b.

In the pattern for ablating the non-spherical component in the second zone 161a, the range for providing the partial ablation with the laser beam of the small zone is limited. Therefore, the surgery time may be shortened .

In the aforementioned preferred embodiments of the configuration of the surgery apparatus 200, the apparatus of the slit scan method has been described as an example. However, the present invention may also be applied to an apparatus of the one shot method or the spot scan method.

In addition, though the determination of the corneal ablation data is performed by the calculating device 150 which is separate from the surgery apparatus 200, it may be performed by the computer 209 included in the surgery apparatus 200. Otherwise, the calculating device 150 may be intended only to calculate the ablation data for improving the aberration of the whole eye, and the ablation data which are divided into those for the zone 160 and the zone 161 may be calculated by the computer 209. That is to say, the size data of the central zone 16O and the like are inputted to the computer 209 along with the data obtained in the calculating device 150. As described above, the computer 209 corrects the ablation data for the whole zone by dividing them into the ablation data for the zone 160 and the zone 161. Incidentally, the same goes for the zones 161a and 161b of the modified embodiment.

As described above, according to the present invention, the corneal ablation surgery may be performed efficiently while improving the aberration of the patient's eye.

The foregoing description of the preferred embodiments of the invention has been presented for purposes of illustration and description. It is not intended to be exhaustive or to limit the invention to the precise form disclosed, and modifications and variations are possible in the light of the teachings described above or may be acquired from practice of the invention. The embodiments chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

What is claimed is:

1. A corneal-ablation-data determining apparatus for obtaining ablation data for corneal ablation surgery to correct a refractive error by ablating a cornea, the apparatus comprising:
input means for inputting measurement data of a patient's eye and size data of an ablation zone; and
calculation means for calculating ablation data including aberration improvement data for the patient's eye based on the inputted measurement data and size data,
wherein the calculation means divides the ablation zone into a central zone and a peripheral zone outside the central zone, then calculates ablation data for a component not being a non-spherical component in the central zone, and calculates ablation data for a non-spherical component in at least part of the peripheral zone, including the aberration improvement data.

2. The corneal-ablation-data determining apparatus according to claim 1, wherein the input means further inputs size data of the central zone.

3. The corneal-ablation-data determining apparatus according to claim 1, wherein a size of the central zone includes a size determined based on a pupil size of the patient's eye in photopic vision.

4. The corneal-ablation-data determining apparatus according to claim 1,
wherein the calculation means further divides the peripheral zone into a first peripheral zone and a second peripheral zone outside the first peripheral zone, then calculates ablation data for the non-spherical component in the first peripheral zone, including the aberration improvement data, and calculates ablation data for the component not being a non-spherical component in the second peripheral zone.

5. The corneal-ablation-data determining apparatus according to claim 4, wherein the input means further inputs size data of the central zone and size data of the first peripheral zone.

6. The corneal-ablation-data determining apparatus according to claim 4,
wherein a size of the central zone includes a size determined based on a pupil size of the patient's eye in photopic vision and a size of the first peripheral zone includes a size determined based on the pupil size in scotopic vision.

7. A corneal surgery apparatus for performing corneal ablation surgery to correct a refractive error by ablating a cornea, the apparatus comprising:
laser irradiation means for irradiating a laser beam to a cornea;
input means for inputting measurement data of a patient's eye and size data of an ablation zone;
calculating means for calculating ablation data including aberration improvement data for the patient's eye based on the inputted measurement data and size data; and
control means for controlling the laser irradiation means based on the obtained ablation data,
wherein the calculating means divides the ablation zone into a central zone and a peripheral zone outside the central zone, then calculates ablation data for a component not being a non-spherical component in the central zone, and calculates ablation data for a non-spherical component in at least part of the peripheral zone, including the aberration improvement data.

8. The corneal surgery apparatus according to claim 7, wherein the input means further inputs size data of the central zone.

9. The corneal surgery apparatus according to claim 7, wherein the calculating means further divides the peripheral zone into a first peripheral zone and a second peripheral zone outside the first peripheral zone, then calculates ablation data for the non-spherical component in the first peripheral zone, including the aberration improvement data, and calculates ablation data for the component not being a non-spherical component in the second peripheral zone.

10. The corneal surgery apparatus according to claim 9, wherein the input means further inputs size data of the central zone and size data of the first peripheral zone.

11. A corneal surgery apparatus for performing corneal ablation surgery to correct a refractive error by ablating a cornea, the apparatus comprising:
laser irradiation means for irradiating a laser beam to the cornea;
input means for inputting ablation data including aberration improvement data for a patient's eye, the ablation data being obtained based on measurement data of the patient's eye;
calculating means for correcting the inputted ablation data; and
control means for controlling the laser irradiation means based on the corrected ablation data,
wherein the calculating means divides an ablation zone into a central zone and a peripheral zone outside the central zone, then calculates ablation data for a component not being a non-spherical component in the central zone, and calculates ablation data for a non-spherical component in at least part of the peripheral zone, including the aberration improvement data.

12. The corneal surgery apparatus according to claim 11, wherein the, input means further inputs size data of the central zone.

13. The corneal surgery apparatus according to claim 11, wherein the calculating means further divides the peripheral zone into a first peripheral zone and a second peripheral zone outside the first peripheral zone, then calculates ablation data for the non-spherical component in the first peripheral zone, including the aberration improvement data, and calculates ablation data for the component not being a non-spherical component in the second peripheral zone.

14. The corneal surgery apparatus according to claim 13, wherein the input means further inputs size data of the central zone and size data of the first peripheral zone.
